(19) United States
(12) Patent Application Publication
(10) Pub. No.: US 2025/0154550 A1
(43) Pub. Date: May 15, 2025

(54) HANGOVER RELIEVER CONTAINING GLUTATHIONE AND ALDEHYDE DEHYDROGENASE

(71) Applicant: PICO ENTECH Co., LTD., Seongnam-Si (KR)

(72) Inventor: Hung Taeck KWON, Seoul (KR)

(73) Assignee: PICO ENTECH Co., LTD., Seongnam-Si, Gyeonggi-Do (KR)

(21) Appl. No.: 19/021,455

(22) Filed: Jan. 15, 2025

Related U.S. Application Data
(62) Division of application No. 17/792,836, filed on Jul. 14, 2022.

(30) Foreign Application Priority Data
Feb. 18, 2020 (KR) 10-2020-0019858
Feb. 17, 2021 (KR) 10-2021-0021473

(51) Int. Cl. C12P 21/02; C12N 1/16; C12N 9/02

(52) U.S. Cl. CPC C12P 21/02; C12N 1/16; C12N 9/0008; C12N 9/0051; C12Y 102/01003; C12Y 108/05001

(57) ABSTRACT

A hangover relieving composition including a dry powder, lysate or extract of yeast that produces glutathione and acetaldehyde dehydrogenase. Further, embodiments relate to a hangover relieving composition containing the dry powder, lysate or extract of Saccharomyces cerevisiae Kwon P-1 KCTC 13925BP and Saccharomyces cerevisiae Kwon P-2 KCTC14122BP and Saccharomyces cerevisiae Kwon P-3 KCTC14123BP yeast that simultaneously produce glutathione and acetaldehyde dehydrogenase.

### BACKGROUND OF THE DISCLOSURE

### Technical Field

The present invention relates to a hangover reliever containing glutathione (GSH) and aldehyde dehydrogenase (hereinafter, ALDH). More specifically, the present invention relates to a hangover reliever containing glutathione and aldehyde dehydrogenase derived from yeasts Saccharomyces cerevisiae Kwon P-1 KCTC 13925BP and Saccharomyces cerevisiae Kwon P-2 KCTC14122BP or Saccharomyces cerevisiae Kwon P-3 KCTC14123BP at the same time.

### Background

Drinking alcohol has been a favorite activity throughout human history, but excessive drinking leads to a hangover that causes physical and mental discomfort, nausea, vomiting, dizziness, thirst, lethargy, drowsiness, headache, and induces abnormalities in the brain nervous system (Alcohol Use Disorder, AUD) (Shao-Cheng Wang et al, 2020), and is emerging as a social problem that induces severe alcohol addiction and even mental panic disorder (Choi Song-sik 2013).

When a person drinks, alcohol is absorbed 5% from the oral cavity, 10-15% from the stomach, 80% from the small intestine and enters the bloodstream, and is broken down by 2-4% in the lungs, 2-4% in the kidneys, 2-6% in sweat, and 90% in the liver.

Alcohol dehydrogenase in the liver is oxidized by alcohol dehydrogenase (ADH), converted to acetaldehyde, and again oxidized and detoxified by aldehyde dehydrogenase (ALDH).

However, 15% to 50% of Asians who lack aldehyde dehydrogenase or genetically have an aldehyde dehydrogenase allele (ALDH2*2) are unable to break down acetaldehyde that is made when drinking alcohol, resulting in blushing Alcohol Flushing Syndrome (Brooks, P J et al. 2009), and it is reported that there is a high probability of getting alcoholism or liver disease due to accumulation of acetaldehyde (Larson, H N et al, 2007). If acetaldehyde is not decomposed and remains in the human body, it causes death and disability due to Alcoholic hepatitis or liver cirrhosis (Gilpin, N W. et al, 2008).

On the other hand, it has been reported that excessive residual of aldehydes (Aldehyde) in the body produced by alcohol intake leads to diseases caused by oxidation such as cardiovascular disease, diabetes, neurodegenerative disease, upper digestive and respiratory tract cancer, radiation dermatitis, Fanconi's anemia, peripheral nerve damage, inflammation, osteoporosis, and aging (Chen et al. 2014).

In addition, it is reported that the scale of socioeconomic loss due to drinking amounts to about 0.5-2.7% of the GDP in most countries, and in the case of Korea, it is reported that the socioeconomic cost of drinking alcohol was estimated to be 14,935.2 billion won in 2000, of which the productivity loss and loss due to diseases, accidents, and hangovers was estimated to be 6.284.5 trillion won (Jung Woo-jin et al. 2006).

In order to solve these social problems, research and experiments on many substances that can reduce the toxicity of ethanol or inhibit the expression of toxicity are in progress, and the results are being developed into various health supplement-related products. Alcohol introduced into the body is absorbed in the stomach or small intestine, enters the blood vessels, is transported to the liver, and is decomposed and detoxified.

Alcohol dehydrogenase (ADH) present in liver cells first oxidizes alcohol to acetaldehyde, and acetaldehyde is decomposed into acetic acid (acetate) by acetaldehyde dehydrogenase (ALDH, aldehyde dehydrogenase) in hepatocytes again, transferred to muscle or adipose tissue throughout the body, and finally decomposed into carbon dioxide and water. Acetaldehyde, the first metabolite of ethanol, is highly reactive and more toxic than ethanol, and is the main cause of hangovers and alcoholic liver disorders.

It has been reported that 19 types of aldehyde dehydrogenase are present in the human body (Marchitti et al. 2007, 2008), and among them, acetaldehyde dehydrogenase 2, which is mainly present in mitochondria, is evaluated to oxidize and remove alcohol-derived acetaldehyde best, as a result of enzymatic engineering analysis, the lowest Km value (˜0.2 μM) was obtained when acetaldehyde was analyzed as an enzyme substrate, compared to when other types of aldehydes were used as substrates.

It is very important to human health to remove aldehyde by most effectively converting acetaldehyde, which is a hangover-causing substance produced by ethanol metabolism in vivo, into acetic acid (Eriksson et al. 1977). In addition, acetaldehyde dehydrogenase 2 is used not only in acetaldehyde but also in the metabolism of aldehydes such as aliphatic aldehydes, aromatic aldehydes, and polycyclic aldehydes to remove toxic substances from the body (Klyosov et al. 1996).

As a representative example, it removes 4-hydroxy-2-nonenal (4-HNE) and malondialdehyde (MDA), which are oxidized aldehyde substances generated in the process of oxidative stress, and serves to remove acrolein from cigarette smoke and automobile exhaust (Chen et al. 2010, Yoval-Sanchez et al. 2012). People with a low expression of the acetaldehyde dehydrogenase 2 enzyme in the human body or whose amino acid residue 487 is mutated from glutamic acid to lysine may have a sensitive reaction to even a small amount of alcohol, such as blushing and flushing. In addition, the concentration of acetaldehyde in the blood is high when drinking alcohol because it cannot be converted (Yoshida et al. 1984).

In particular, those with ALDH2-2, a homozygous for acetaldehyde dehydrogenase 2, are known to be vulnerable to drinking, and this genetic mutation is rarely seen in Westerners, but is found in 50% of the total population in Koreans, Chinese, and Japanese (Brooks et al. 2009).

As for R&D on aldehyde dehydrogenase 2, the importance of R&D for aldehyde dehydrogenase 2 is being emphasized as research on promoters and inhibitors of aldehyde dehydrogenase 2 in the body has been actively studied for medical purposes (Budas et al. 2009, Chen et al. 2014, M.zel et al. 2018), there is still a lack of research on breeding microorganisms for overproduction of aldehyde dehydrogenase 2 or development of mass production technology.

The purpose of the development of a strain that produces an excess of aldehyde dehydrogenase 2 is to express human aldehyde dehydrogenase 1 and 2 proteins using a protein expression system using E. coli as a host. It has been reported that about 30% of these are expressed as active soluble enzymes to produce 2-4 mg/L of protein (Zheng et al. 1993), and in the case of Rat aldehyde dehydrogenase 2, it was reported that 95% was expressed as an active soluble protein, but very little protein of 1 to 2 mg/L was produced (Jeng et al. 1991).

However, there are no reports of cases of increasing the production of acetaldehyde dehydrogenase 2 using a mutagenesis method that is easy to use due to small legal restrictions. Therefore, there is an urgent need to develop microorganisms with highly active aldehyde dehydrogenase 2 by mutagenesis to expand the range of use of acetaldehyde dehydrogenase 2.

Ethanol absorbed in the body is oxidized to acetaldehyde by the enzyme ADH (alcohol dehydrogenase), and in the decomposition/oxidation process of acetaldehyde produced by alcohol oxidation, an enzyme called ALDH (aldehyde dehydrogenase) acts to decompose it into carbon dioxide gas and water, and it is discharged outside the body.

ALDH decomposes not only acetaldehyde, but also Nonenal (4-hydroxy-2-nonenal), HNE (4-hydroxy-trans-2-nonenal), malondialdehyde, DOPAL (3,4-dihydroxy-phenylacetaldehyde), DOPEGAL (3,4-dihydroxy-phenylglycolaldehyde), 5-HIAL (5-hydroxy indole-acetaldehyde), and retinaldehyde, etc. (Arnold SL et al, 2015).

These various types of aldehydes destroy DNA in the human body (Garaycoechea, J I et al, 2018), and cause serious diseases by degrading the mitochondria which is an important energy-producing organ of the cell (Mitochondrial dysfunction) (Gomes, K M et al, 2014).

For the decomposition of these various aldehydes, ALDH mainly from the yeast Saccharomyces is widely used. According to the Genome Database, in the genome of yeast, about six types of ALDH (Datta S. et al, 2017) are known in the genus Saccharomyces.

Among them, ALDH2 is structurally similar to human ALDH in the binding site of the coenzyme NAD (Mukhopadhyay, A. et al, 2013), uses NAD as a coenzyme, and acts in the mitochondria, but also in the cytoplasm of organisms other than the mitochondria in yeast. As for the specific activity of the enzyme, yeast ALDH (y ALDH) is more than 20 times higher than that of human ALDH (h ALDH) (M.-F. Wang et al, 2009), so a high effect can be expected when used in the human body.

Conventional rice-derived yeast ALDH produced by solid culture in rice has the advantage of being easy to purify, but there is a limit to the commercial mass production of hangover relievers due to the low ALDH production yield. In order to improve this problem, a method for mass production of rice-derived yeast ALDH has emerged. A method for making recombinant yeast by securing the ALDH gene is described in Korean Patent Publication No. 10-2005-0052664 (PCT/EP2003/01049).

A technique for recombination of an aldehyde dehydrogenase gene (ALDH gene) derived from yeast is described in Korean Patent Registration No. 10-1664814. A method for producing an ADH enzyme that oxidizes alcohol by genetic recombination is described in Korean Patent Application No. 10-2020-0045978.

On the other hand, various efforts are being made to develop an activator that activates the ALDH enzyme in the human body using various health food materials to prevent hangovers caused by alcohol intake, relieve hangover, and prevent liver damage (U.S. Pat. Nos. 10,406,126 10,406,126 B2 (2019), US Pub. NO US2020/0237716 A1 (2020)).

In Korea, as an activator, herbal extracts (Korean Patent Application No. 10-2020-0142768) prepared alone or in combination with herbal preparations such as chrysanthemum, licorice, galgeun, dermis, and Heotgae are known.

In addition, Korean Patent Registration No. 10-0696589 discloses a hangover relieving composition containing Hwangtae, Heotgae tree, mistletoe extract and arrowroot ingredients, and Korean Patent Publication No. 10-2012-0123860 discloses the use of a reducing agent glutathione by providing a hangover relieving composition comprising turmeric, alder tree, oriental raisin tree pedicel, spiny hornwort concentrate, unembroidered soybean ferment extract, milk thistle, and glutathione.

However, most of the patented technologies so far have focused more on relieving hangovers than preventing hangovers, and in many cases the effect of relieving hangovers is insignificant. Therefore, there is a need in the art to develop a hangover relief composition containing ALDH that can directly and quickly detoxify acetaldehyde, which is the fundamental problem of a hangover phenomenon.

The present inventors have developed a hangover relieving composition containing glutathione and ALDH, which acts quickly in the body to rapidly decompose alcohol and aldehydes and rapidly decomposes aldehyde products that cause various reactive oxygen species (ROS) generated during human metabolic processes, and which can contribute to the protection of human physiological functions as well as a hangover by maintaining its effect in the human body.

An object of the present invention is to provide a novel hangover relieving composition that contains sufficient amounts of ALDH enzyme and glutathione to ensure rapid and continuous efficacy of removing aldehydes and toxins from the body. The hangover relieving composition according to the present invention maintains a variety of tolerable aldehyde detoxification activities in the human body as well as aldehyde detoxification activity in the digestive system.

On the other hand, Glutathione (γ-L-glutamyl-L-cysteinylglycine, GSH) is a tripeptide composed of three amino acids, glutamate, cysteine, and glycine as a physiologically active substance present in cells, and it is present in the cells of animals, plants, and microorganisms at a concentration of 0.1 to 10 mM, and accounts for more than 90% of the total non-proteinaceous active ingredients of the cells.

In vivo, glutathione is known to play an important antiviral role by causing an increase in immune activity through the production of white blood cells and play an important role in detoxification by acting as a substrate for GST (glutathione S-transferase) and combining toxic substances such as xenobiotics which are harmful to the living body in the form of conjugation.

In addition, glutathione prevents necrosis by damaging cell membranes, nucleic acids, and cellular structures through oxidation within the cell and plays a role in alleviating the toxicity of reactive oxygen species (ROS), the cause of aging. At this time, reactive oxygen species are formed in various biological metabolic reactions, and include superoxide, peroxide, hydroxyl radicals, etc., and can be divided into an endogenous reactive oxygen species produced as a biological metabolite of a substance and an exogenous reactive oxygen species such as tobacco and radioactivity.

Oxidative stress caused by reactive oxygen species can impair cognitive function (Liu et al. 2002), cause male infertility by destroying sperm DNA (Wright et al. 2014), and cause cancer by damaging cellular proteins, lipids, and nucleic acids and acts as a causative factor of various diseases and aging by lowering physiological functions. Therefore, antioxidants are very important for our body to prevent diseases, improve immunity, and prevent aging, and glutathione's function as an antioxidant in cells has attracted attention in many medical fields, including enzymology, pharmacology, therapeutics, toxicology, endocrinology, and microbiology.

Although such glutathione is basically synthesized in the body, the absolute content of glutathione in the human body decreases as abnormal conditions such as disease outbreak, weakened immune system, and aging progress, which deteriorate health. Therefore, glutathione supplied from the outside can remove reactive oxygen species in cells to maintain health and slow aging. Due to the physiologically active factors of glutathione in the human body, glutathione is currently used for food, cosmetics, feed, and pharmaceuticals, and its usage is gradually increasing.

On the other hand, the production of glutathione is currently produced using edible microorganisms, but since the intrinsic content of glutathione that microorganisms can produce is very low, research has been conducted to increase the glutathione content of microorganisms through mutation and recombination techniques, and to mass-produce high-content glutathione-producing strains using fermentation techniques.

Thus, the development of strains with a high glutathione content is to develop a fundamental material that increases economic value, and enables glutathione to have a competitive edge in the market that allows it to be widely used in health foods, pharmaceuticals, and feeds. etc.

However, strain development by genetic recombination technology cannot be free from the various problems of GMO that are currently an issue, so the scope of its usage is limited. However, the performance-improved strain by mutation technology has relatively few limitations, so it is relatively easy to develop for various uses. Therefore, breeding technology for strain producing high content of glutathione using mutation technology is suitable for production of glutathione used as an active ingredient in food or medicine.

As described above, the efficiency is high when glutathione and aldehyde dehydrogenase are used simultaneously to remove chemicals such as reactive oxygen species and various aldehydes among various harmful substances accumulated in the human body, though strains that simultaneously mass-produce glutathione and aldehyde dehydrogenase have not been commercialized until the present day.

### SUMMARY

An aspect of the present disclosure provides a hangover remedy composition, a strain capable of producing aldehyde dehydrogenase and glutathione at the same time with high efficiency is used to make a mutant strain using a primary chemical mutation method, and secondary selection factor adaptation mutants were selected and developed.

Mutant strains that simultaneously overproduces glutathione and acetaldehyde dehydrogenase 2 are reported as GRAS (Generally Recognized As Safe) with no problems for food, health food, feed, cosmetic and medical use, and wild Saccharomyces cerevisiae is selected and used, which is already known as a strain that produces both glutathione and aldehyde dehydrogenase although production efficiency is low.

In this way, the production capacity of glutathione is increased through mutation, and at the same time, a new improved strain, Saccharomyces cerevisiae sp. with increased aldehyde dehydrogenase production capacity, was prepared, and this strain dried powder, lysate or ALDH-containing extract was prepared to complete the hangover reliever of the present invention.

According to an exemplary embodiment of the present disclosure, the active ingredient of the hangover remedy composition of the present invention is described in detail in Korean Patent Application No. 10-2020-0019858, and is a dry powder, lysate, or ALDH-containing extract of Saccharomyces cerevisiae Kwon P-1 (KCTC13925BP), Saccharomyces cerevisiae Kwon P-2 (KCTC14122BP) or Saccharomyces cerevisiae Kwon P-3 (KCTC14123BP), which have been deposited with the International Depositary Organization (KCTC).

The present inventors have invented a new fermentation process that dramatically increases the production yield of ALDH as a two-step process including performing the step of fermenting the first liquid phase by using three strains, such as Saccharomyces cerevisiae Kwon P-1 (accession number: KCTC13925BP), which have high ALDH production ability and high glutathione production ability by mutation, alone or in combination, and then adding a liquid fermentation product to rice fermented powder to proceed with secondary solid-phase fermentation.

In addition, in the new fermentation process of the present invention, one of three strains such as Saccharomyces cerevisiae Kwon P-1 (accession number: KCTC13925BP) or a mixed strain thereof is used, and the first liquid-state fermentation step and the second solid-state fermentation step is in progress, then it is possible to complete a fermentation process capable of simultaneously producing glutathione and ALDH enzymes, which are powerful reducing agents, in high yield.

Saccharomyces cerevisiae Kwon P-1 (accession number: KCTC13925BP), which was cultured in large quantities, was again inoculated into rice, the Saccharomyces cerevisiaestrain, which produces excessive amounts of glutathione and acetaldehyde dehydrogenase by solid-phase fermentation, is cultured on a larger scale in a two-step process, and a hangover relieving composition of the present invention was prepared containing the dry powder, lysate or extract powder of the strain.

### BRIEF DESCRIPTION OF THE DRAWINGS

FIG. 1 is a graph showing changes in blood acetaldehyde content of experimental animals according to administration of a composition of the present invention.

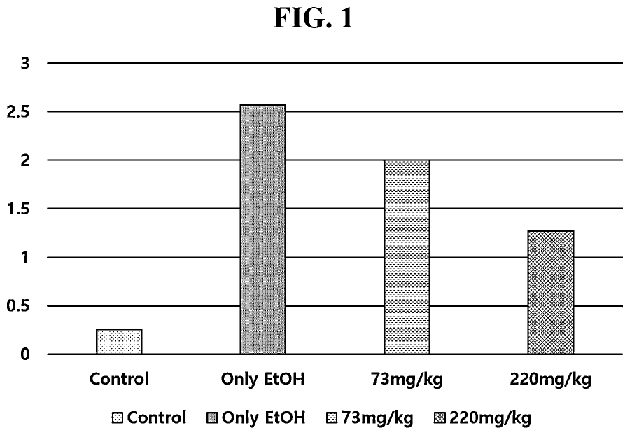

FIG. 2 is a graph showing changes in blood acetaldehyde content of experimental animals according to administration of the composition of the present invention.

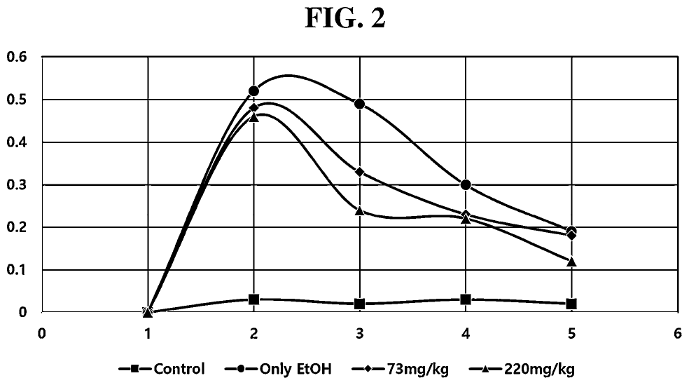

### DETAILED DESCRIPTION OF THE EMBODIMENTS

Hereinafter, the configuration and effects of the present invention will be described in more detail through the following examples. These examples are only for illustrating the present invention, and the scope of the present invention is not limited by these examples. Hereinafter, the configuration and effects of the present invention will be described in more detail through examples. The following examples are only for illustrating the present invention, and the scope of the present invention is not limited by these examples.

### [Example 1] Preparation of Yeast Lysate Containing Glutathione and ALDH

### Example 1-1: Saccharomyces cerevisiae Yeast Fermentation Process Containing Glutathione and ALDH

The ALDH-containing Saccharomyces cerevisiae yeast seed was fermented and cultured for 24 hours in an incubator at 160 rpm and 30° C. using YPD medium (yeast extract, peptone, and glucose containing medium) in a 200 mL flask, the culture was carried out for 72 hours through a 5 L fermenter (Marado-05D-PS, CNS, Korea). After completion of the culture, the yeast was centrifuged using a high-speed centrifuge (Supra R22, Hanil, Korea).

### Example 1-2: Preparation of Yeast Lysate Containing Glutathione and ALDH

The centrifuged ALDH-containing yeast was frozen in a cryogenic freezer (CLN-52U, Nihon freezer, Japan) for 2 days, and then freeze-dried for 2 days in a freeze dryer (FDU-7006, Operon, Korea). After dissolving 3 g of lyophilized yeast powder in 50 mL phosphate-buffered saline (PBS) containing a protease inhibitor (A32955, Thermo fisher, USA), 10 g of 0.5 mm glass beads for cell disruption (11079105, Biospec) were put into the bead homogenizer (Mixer Mill MM400, Retsch, Germany) for 2 minutes a total of 3 times to disrupt the yeast. After centrifugation using a high-speed centrifuge (Supra R22, Hanil, Korea), only the supernatant was separated and freeze-dried for 2 days with a freeze dryer (FDU-7006, Operon, Korea).

### [Example 2] Mass Production of Saccharomyces cerevisiae Strain by Two-Step Fermentation Process

Saccharomyces cerevisiae KwonP-1 strain (KCTC13925BP) was inoculated into YPD medium containing 2% peptone, 1% yeast extract, and 2% glucose and cultured at 30° C. and fermented at 200rpm, 1 vvm in a fermentor (Fermentor, Cobiotech) until the OD600 nm value reached 50.

The recovered cells are mixed with the already sterilized rice fermented powder at a ratio of 10%, the moisture content is adjusted to 60%, and the solid phase is cultured for 2 days at 30° C. and then dried at 50° C. to adjust the final moisture content to 7%, and yeast-fermented rice fermented powder was prepared.

The fermentation composition of the present invention thus prepared contained a maximum of 600 units/g of ALDH. Considering that the rice fermented powder by the wild-type Saccharomyces cerevisiae yeast strain known to date generally contains about 2 unit/g of ALDH, it was confirmed that the ALDH content of the fermented composition of the present invention was increased by about 300 times.

Table 1 shows the results of evaluating the acetaldehyde decomposition ability of the compositions (1 to 4) of the present invention prepared by the two-stage fermentation process of the present invention for 5 minutes.

TABLE 1

| | Reaction | | Suspension | | |
|---|---|---|---|---|---|
| | time | NADPH | vol. | Unit/g | |
| | (m) | (mM) | (ml) | powder | Average |
| One | 5 | 1.732 | 0.20 | 693.0 | 681.4 |
| One | 5 | 1.675 | 0.20 | 669.8 | |
| 2 | 5 | 1.722 | 0.20 | 688.9 | 682.2 |
| 2 | 5 | 1.689 | 0.20 | 675.6 | |
| 3 | 5 | 1.770 | 0.20 | 707.9 | 702.1 |
| 3 | 5 | 1.741 | 0.20 | 696.3 | |
| 4 | 5 | 1.639 | 0.20 | 655.8 | 665.7 |
| 4 | 5 | 1.689 | 0.20 | 675.6 | |

The ALDH coenzyme NAD was added as an enzyme activator to the dry pulverized powder of the fermentation composition of the present invention prepared in this way, and citric acid, magnesium stearate, DL methionine, vitamin C and lactic acid bacteria (Lactobacillus plantium 10 7/g), zinc oxide, and silicon dioxide were added to prepare a hangover reliever of the present invention.

Through animal testing of the hangover reliever of the present invention, the concentration of acetaldehyde in the blood was measured after alcohol intake, and the hangover reliever of the present invention significantly and rapidly reduced the concentration of acetaldehyde in the blood compared to the conventional hangover reliever.

In addition, for the human clinical trial of the hangover reliever of the present invention, voluntary clinical trial volunteers were subjected to a genomic test to divide the volunteers into an experimental group, which is an ALDH2 possessing group that is able to decompose aldehydes, and an experimental group, which is genetically deficient in the ability to decompose aldehydes and has a ALDH2*2 mutant gene.

As a result of the hangover relieving ability test in the human body for 15 hours, significant differences in aldehyde degradation ability were confirmed in both the ALDH2 retention test group and the ALDH2*2 gene mutation test group. The hangover reliever of the present invention was able to efficiently remove acetaldehyde in both experimental groups. In particular, it efficiently removes aldehydes even in the ALDH 2*2 gene mutation experimental group, where it is difficult to degrade aldehydes, it was confirmed that the hangover reliever of the present invention is effective in decomposing aldehydes and relieving hangovers due to the content enhancement of ALDH and Glutathione.

### [Example 3] Measurement of Hangover Relief Effect of the Composition of the Present Invention

### Example 3-1: Animal Test for Changes in Blood Acetaldehyde Over Time

Table 2 shows the animal test results on the temporal change of acetaldehyde in blood after ethanol administration.

TABLE 2
Blood Acetaldehyde (mg/L)

| | 0 hr | 1 hr | 3 hr | 5 hr | 8 hr |
|---|---|---|---|---|---|
| Control group | 0 | 0.03 | 0.02 | 0.03 | 0.02 |
| Ethanol administration group | 0 | 0.52 | 0.49 | 0.3 | 0.19 |
| Ethanol and the composition | 0 | 0.48 | 0.33 | 0.23 | 0.18 |
| administered at 73 mg/kg | | | | | |
| Ethanol and the composition | 0 | 0.46 | 0.24 | 0.22 | 0.12 |
| 220 mg/kg administered group | | | | | |

Table 3 shows the results of the animal test of the cumulative amount of aldehydes in the blood (mg/L·hr).

TABLE 3

| | Accumulated | |
|---|---|---|
| | amount of | |
| | acetaldehyde | Decrease |
| | in the blood | rate |
| Control group | 0.26 | |
| Ethanol administration group | 2.57 | |
| Ethanol and the composition | 2 | −22% |
| 73 mg/kg administered group | | |
| Ethanol and the composition | 1.27 | −51% |
| 220 mg/kg administered group | | |

### Example 3-2: Analysis of Ethanol and Isetaldehyde in Blood of Human Clinical Trial Volunteers

Volunteers for the human clinical trial were selected from 43 healthy adult males between their 20s and 40s who can drink soju based on an average alcohol content of 20 degrees at a time of drinking, and the clinical trial was conducted once a week on Friday at 17:00 for a total of 4 weeks. Clinical trials were conducted through a camp for a total of 15 hours at 8 o'clock the day after admission to the hospital, and a total of 23 patients finally completed the clinical trials due to personal circumstances during the clinical process.

On the first day of the camp, the amount of change in alcohol concentration and acetaldehyde concentration was measured after drinking 10 glasses of soju, which is a blood alcohol metabolism measurement in each time period, on the second day of the camp, the amount of changes in blood alcohol metabolism was measured after drinking 10 glasses of soju 30 minutes after taking 73 mg/kg of the composition of the present invention, and on the second day of the camp, the amount of change in blood alcohol metabolism was measured after drinking 10 glasses of soju 30 minutes after taking 220 mg/kg of the composition of the present invention.

In the group taking the composition containing 500 mg/day of the double-fermented dry powder and 1500 mg of fermented rice powder of the present invention, the blood concentration of Acetaldehyde, which is a causative agent of hangovers and a strong carcinogen in the body, was significantly reduced in a dose-dependent manner compared to the alcohol alone group. In addition, the residual amount of blood alcohol was also significantly decreased in a dose-dependent manner with the composition of the present invention.

Table 4 shows the decrease in blood alcohol concentration of human clinical trial volunteers.

TABLE 4

| | Alcohol | Alcohol | | Peak |
|---|---|---|---|---|
| | accumulation | reduction | Decrease | Cmax |
| | (g · hr/dL) | (%) | rate | (g/L) |
| Alcohol only administration group | 30.852 | 100% | | 7.583 |
| Ethanol and composition | 29.693 | 96.2% | −3.8% | 5.548 |
| 73 mg/kg administration group | | | | |
| Ethanol and composition | 25.271 | 85.7% | −14.9% | 4.18 |
| 220 mg/kg administration group | | | | |

Table 5 shows the decrease in the residual amount of acetaldehyde in the blood of volunteers in human clinical trials.

TABLE 5

| | Residual | | | |
|---|---|---|---|---|
| | amount of | | | |
| | acetaldehyde | Residue | | Peak |
| | in blood | reduction | Decrease | Cmax |
| | (mg · hr/dL) | (%) | rate | (mg/dL) |
| Ethanol administration group | 13.02 | 100% | | 1.65 |
| Ethanol and composition | 9.39 | 72.1% | 27.9% | 1.2 |
| 73 mg/kg administration group | | | | |
| Ethanol and composition | 5.22 | 68.0% | −32.0% | 1.3 |
| 220 mg/kg administration group | | | | |

### Example 3-3: Test for Confirming Changes in Ethanol and Acetaldehyde According to ALDH Gene Mutation

For the recruitment of volunteers for the human clinical trial, 43 adult males in their 20s to 40s who can drink soju based on an average alcohol content of 20% at a time of drinking were selected. For a total of 4 weeks, a clinical trial was conducted through a camp for a total of 15 hours at 8 o'clock the day after admission to the hospital, and a total of 23 patients finally completed the clinical trials due to personal circumstances during the clinical process.

Among them, about 22 people participated in the alcohol metabolism-related genetic test, and obtained consent for the experiment and information use, and a total of 22 people performed three types of genomic test, ADH1B (Alcohol dehydrogenase 1B), ALDH2 (Aldehyde dehydrogenase 2), and CPY2E1 P450, which are involved in alcohol metabolism in vivo, it was confirmed that the blood concentration of Acetaldehyde, a substance that causes hangovers and a strong carcinogen in the body, decreased in a dose-dependent manner in both the ALDH2 non-mutant group and the ALDH2*2 mutant group compared to the alcohol only administration group.

In the case of the ALDH2*2 gene mutation group, it is known that even a small amount

of alcohol shows a very high blood acetaldehyde concentration, the effect of reducing blood aldehydes has never been observed or reported through conventional hangover relieving drinks or conventional hangover relieving foods and drugs. However, in the case of administration of the hangover relieving composition of the present invention, the effect of reducing blood Acetaldehyde in the ALDH2*2 gene mutant group is very remarkable.

Table 6 shows the alcohol content (g hr/L) of the normal ALDH gene carrying group and the ALDH gene mutant group.

TABLE 6

| | ALDH | Normal |
|---|---|---|
| | gene | ALDH gene |
| | mutant | carrying |
| | group | group |
| Ethanol administration group | 42.55 | 28.13 |
| Ethanol and composition | 36.13 | 29.86 |
| 73 mg/kg administration group | | |
| Ethanol and composition | 40.87 | 24.94 |
| 220 mg/kg administration group | | |

Table 7 shows the average blood acetaldehyde content (g hr/L) of the normal ALDH gene carrying group and the ALDH gene mutant group.

TABLE 7

| | ALDH | Normal |
|---|---|---|
| | gene | ALDH gene |
| | mutant | carrying |
| | group | group |
| Ethanol administration group | 10.56 | 13.47 |
| Ethanol and composition | 8.78 | 7.57 |
| 73 mg/kg administration group | | |
| Ethanol and composition | 8.43 | 3.92 |
| 220 mg/kg administration group | | |

### [Example 4] Toxicity Test of the Hangover Relieving Composition of the Present Invention

### Example 4-1. Preparation of Laboratory Animals

As experimental animals, female and male ICR mice (7 weeks old) were received and acclimatized for 7 days. During the acclimatization period, general symptoms were observed, and only healthy animals were used for the test. Feed and water were ingested ad libitum, and group separation was performed so that there were 5 males and 5 females in each group based on the average body weight of about 20 g the day before oral administration.

### Example 4-2 Administration of the Hangover Relieving Composition of the Present Invention

The test substance was prepared by dissolving in physiological saline so that the doses of the test animals were 0, 750, 3000, and 5000 mg/Kg, respectively, based on the content of the yeast lysate containing GSH and ALDH of the present invention. The standard of administration dose complied with the Korea National Toxicology Program (KNTP) toxicity test manual of the Ministry of Food and Drug Safety, and the maximum applied dose 5000 mg/Kg guided by the KNTP manual was applied as the maximum concentration of this experiment. Samples prepared for each group were orally administered once to each test animal, and physiological saline was administered to the normal group (G1).

### Example 4-3. Observation and Autopsy

Symptoms were observed at least once a day from the date of acquisition to the day of autopsy for all animals in the test group, and symptoms were observed for 7 days after oral administration. After the type of symptom observation, an autopsy was performed, and changes in each organ were visually observed at the time of autopsy.

As a result of a single-dose toxicity test using the yeast lysate containing glutathione and ALDH of the present invention in mice, no mortality was observed for 7 days, and no peculiarities such as weight gain and feed intake, etc. were found at a concentration of up to 5000 mg/kg. Also, no unusual findings were found in the autopsy results after the observation was completed.

### [Accession Number]

Name of deposit institution: Korea Research Institute of Bioscience and Biotechnology
Accession number: KCTC13925BP
Deposit date: 20190822
Name of deposit institution: Korea Research Institute of Bioscience and Biotechnology
Accession number: KCTC14122BP
Deposit date: 20200130
Name of deposit institution: Korea Research Institute of Bioscience and Biotechnology
Accession number: KCTC14123BP
Deposit date: 20200130

What is claimed is:

1. A Saccharomyces cerevisiae strain mass culture method, comprising:
(a) culturing the Saccharomyces cerevisiae strain in a liquid medium, and
(b) culturing the Saccharomyces cerevisiae strain further cultured in the (a) step in a solid medium again.

2. The Saccharomyces cerevisiae strain mass culture method of claim 1, wherein the liquid medium is YPD medium containing peptone, yeast extract and glucose, and wherein the solid medium is anyone selected from a group consisting of rice, barley, wheat, corn, and soybeans, or a mixture thereof.

3. The Saccharomyces cerevisiae strain mass culture method of claim 1, wherein the Saccharomyces cerevisiae strain is selected from a group consisting of Saccharomyces cerevisiae Kwon P-1 (KCTC13925BP), Saccharomyces cerevisiae Kwon P-2 (KCTC14122BP), and Saccharomyces cerevisiae Kwon P-3 KCTC14123BP.
